(19) United States
(12) Patent Application Publication
(10) Pub. No.: US 2009/0077678 A1
(43) Pub. Date: Mar. 19, 2009

(54) TRANSGENIC MOLLUSK AND METHOD FOR PRODUCING THE SAME

(76) Inventors: Keizaburo Miki, Naka-gun (JP); Johji Miwa, Yokohama-shi (JP); Nozomu Isowa, Shima-gun (JP)

Correspondence Address: BIRCH STEWART KOLASCH & BIRCH, PO BOX 747, FALLS CHURCH, VA, 22040-0747, US

(21) Appl. No.: 12/272,471

(22) Filed: Nov. 17, 2008

Related U.S. Application Data
(63) Continuation of application No. 11/094,447, filed on Mar. 31, 2005, now abandoned.
Application No. 11/094,447 is a continuation of application No. 09/914,190, filed on Mar. 20, 2002, now abandoned.

(30) Foreign Application Priority Data
Feb. 25, 1999 (JP) 48444/99

(51) Int. Cl. A01K 67/00

(52) U.S. Cl. 800/13; 800/21

(57) ABSTRACT

A transgenic mollusk which can express a desired foreign gene, and a method for producing the same are disclosed. The mollusk is a transgenic mollusk into which a desired foreign gene (excluding a gene giving resistance to a virus) is introduced, which expresses the foreign gene. This transgenic mollusk can be produced by microinjecting into gonad of male and/or female of mollusk a recombinant vector into which a desired foreign gene to be introduced or a nucleic acid containing the foreign gene is inserted; crossing the male and female to produce individuals of first generation; and selecting therefrom (an) individual(s) which express(es) the desired gene.

This application is a Continuation of copending U.S. application Ser. No. 11/094,447, filed on Mar. 31, 2005, which is a Continuation of U.S. application Ser. No. 09/914,190, filed on Mar. 20, 2002, now abandoned, and for which priority is claimed under 35 U.S.C. § 120. Application Ser. No. 09/914,190 is the national phase of PCT International Application No. PCT/JP00/01060 filed on Feb. 24, 2000 under 35 U.S.C. § 371. This application also claims priority under 35 U.S.C. § 119(a) of Patent Application No. 48444/99 filed in Japan on Feb. 25, 1999. The entire contents of each of the above-identified applications are hereby incorporated by reference.

### TECHNICAL FIELD

The present invention relates to a transgenic mollusk and a method for producing the same.

### BACKGROUND ART

By the conventional pearl culture, only several kinds of naturally-occurring pearls can be produced, and their color tones are modified by staining or the like after production of the pearls. Such modification of color after production of the pearls is likely to fade and it is almost impossible to change the color tone to the desired one because the nacreous layer is hard.

### DISCLOSURE OF THE INVENTION

Providing a transgenic pearl shell which has an ability to produce a colored pearl is advantageous in the field of pearl culture. However, to say nothing of a transgenic pearl shell having such a useful property, in the entire phylum Mollusca, a transgenic mollusk which can express a desired foreign gene has not been produced.

Accordingly, an object of the present invention is to provide a transgenic mollusk which can express a desired foreign gene and a method for producing the same.

The present inventors intensively studied to succeed in producing a transgenic mollusk which expresses a desired foreign gene by microinjecting into gonad of male and female mollusks a recombinant vector into which a desired foreign gene to be introduced or a nucleic acid containing the foreign gene is inserted, and crossing the male and female; or by introducing into fertilized eggs or embryos of a mollusk to be transformed a recombinant vector into which a nucleic acid including a promoter having a promoter activity in said mollusk and said desired gene located at a downstream region of said promoter is inserted, thereby completing the present invention.

That is, the present invention provides a transgenic mollusk into which a desired foreign gene (excluding a gene giving resistance to a virus) is introduced and which expresses the foreign gene. The present invention also provides a method for producing the above-mentioned transgenic mollusk according the present invention, comprising microinjecting into gonad of male and/or female of mollusk a recombinant vector into which a desired foreign gene to be introduced or a nucleic acid containing the foreign gene is inserted; crossing the male and female to produce individuals of first generation; and selecting therefrom (an) individual(s) which express(es) the desired gene. The present invention further provides a method for producing the above-mentioned transgenic mollusk according to the present invention, comprising introducing into unfertilized eggs, fertilized eggs or embryos of a mollusk to be transformed a recombinant vector into which a nucleic acid including a promoter having a promoter activity in the mollusk and the desired gene located at a downstream region of the promoter and functionally linked to the promoter is inserted; developing the unfertilized eggs, fertilized eggs or embryos to individuals; and selecting therefrom (an) individual(s) which express(es) the desired gene.

By the present invention, a transgenic mollusk which can express a desired foreign gene was first provided. The present invention made it possible to produce various mollusks, such as a pearl shell yielding a colored pearl, which are industrially useful.

### BEST MODE FOR CARRYING OUT THE INVENTION

The transgenic mollusk according to the present invention may be any animal belonging to the phylum Mollusca. Preferred examples of the mollusk include shellfishes belonging to the class Bivalvia or Gastropoda, especially, pearl shells such as Pinctada fucata Martensii, Pinctada maxima and Pinctada margaritifera.

The desired foreign gene to be introduced into the mollusk may be any gene which can give the character to be given to the mollusk (excluding a gene giving resistance to a virus). Preferred examples thereof include, in cases where the mollusk is a pearl shell, genes relating to coloring such as green fluorescence protein (GFP) gene, anthocyanin gene, fluorescent luciferase gene, β-galactosidase gene and phosphatase gene. The term “genes relating to coloring” includes, as apparent from the above-mentioned examples, not only genes encoding pigments (including fluorescent pigments), but also genes encoding substances which participate in pigment-formation reactions in the body, such as genes encoding enzymes which catalyze reactions forming pigments in the body.

The transgenic mollusk according to the present invention may be produced as follows: In the first method, a recombinant vector into which a desired foreign gene to be introduced or a nucleic acid containing the foreign gene is inserted is microinjected into gonad of male and/or female of mollusk; the male and female are crossed to produce individuals of first generation; and (an) individual(s) which express(es) the desired gene is(are) selected therefrom.

What inserted into the recombinant vector to be microinjected may be the desired foreign gene alone or may be a nucleic acid containing the foreign gene. Examples of such a nucleic acid include those containing a promoter which shows promoter activity in the mollusk cells, which promoter is located at an upstream region of the foreign gene, and fusion genes in which the foreign gene is fused with another gene. Examples of the genes to be fused with the desired foreign gene include, in cases where the mollusk is a pearl shell, nacreous layer protein gene, prism layer skeleton protein gene and calcium carbonate-crystallizing enzyme gene. Examples of the vector include vectors for animal cells such as adenovirus vectors, retrovirus vectors and the like. These vectors are commercially available, and commercially available vectors may be employed.

The method for microinjecting the above-mentioned recombinant vector to the gonad of a mollusk will now be described. Basically, the microinjection may be carried out by directly injecting a solution containing the recombinant vector into the gonad through an injection needle. The medium of the solution for microinjection may be a buffer such as TE buffer. The concentration of the recombinant vector in the solution may preferably be about 2 to 200 μg/ml, more preferably about 5 to 10 μg/ml. The amount of the solution to be injected may preferably be about 10 to 50 μl per site, and it is preferred to inject the solution into 2 to 4 sites in a gonad in case of either ovary or testis.

After the microinjection, the mollusk is left to stand at 10 to 25° C., preferably at 15 to 20° C. for 24 to 72 hours, preferably 24 to 48 hours, and then the male and the female which received the microinjected solution are crossed. Although the crossing may be carried out by natural crossing, to securely attaining the crossing with high reproducibility, it is preferred to carry out the crossing by artificial fertilization. Artificial fertilization may be carried out basically by adding the sperms from the testis which underwent the microinjection to mature eggs in the ovary of the female that underwent the microinjection. Although it is preferred to conduct the microinjection on the gonads of both of the male and female to be crossed, a transgenic mollusk may be produced even if the gonad of only one of the male and female is subjected to the microinjection. The artificial fertilization per se of the mollusk may be carried out by the method described in Dev Biol 1994, 163(1): 162-174 (this paper is herein incorporated by reference) or the like.

Growing individuals from the fertilized eggs may easily be carried out by incubating the fertilized eggs in sea water or artificial sea water at a temperature within the growing temperature range of the mollusk.

Transgenic individuals are then selected from the obtained individuals. This may be carried out by checking whether the desired foreign gene introduced exists in the mollusk cells by Southern blotting, and further checking whether the foreign gene is expressed in the mollusk cells by Northern blotting. Southern blotting and Northern blotting per se, as well as methods for preparing the samples therefor are well-known in the art, and are described in, for example, Nakayama and Nishigata, “Bio Experiments Illustrated-\textcircled{2} Base of Gene Analysis”, Shujunsha (1995).

To establish a transgenic line, it is preferred to cross the male and female of the individuals of the first generation, which were confirmed to be transgenic, thereby obtaining the individuals of the second generation, and to select the transgenic individuals therefrom in the same manner as mentioned above. Further, by producing the third or more generation, the transgenic line may be established more securely.

In the second method for producing the transgenic mollusk, a recombinant vector into which a nucleic acid including a promoter having a promoter activity in the mollusk and the desired gene located at a downstream region of the promoter and functionally linked to the promoter is inserted is introduced into unfertilized eggs, fertilized eggs or embryos of a mollusk to be transformed; the unfertilized eggs, fertilized eggs or embryos are developed to individuals; and (an) individual(s) which express(es) the desired gene is (are) selected therefrom.

The promoter showing promoter activity in the mollusk to be transformed may be any promoter which shows promoter activity in the mollusk to be transformed. Examples of the promoter include actin gene promoter and heat shock protein gene promoter, but the promoter is not restricted thereto. The term “functionally linked” in the phrase “the desired gene located at a downstream region of the promoter and functionally linked to the promoter” means that the desired gene is linked to the promoter such that the reading frame thereof is so adjusted as to be controlled by the promoter. In this case, it is possible to express the foreign gene as a fused protein by ligating the desired foreign gene to the downstream end of a structural gene or a fragment thereof such that the reading frame of the desired gene is coincide with that of the structural gene or a fragment thereof, which structural gene or a fragment thereof is functionally linked to the promoter. The method for functionally link a structural gene to a promoter at a site downstream of the promoter is well-known in the art. The recombinant vector may be prepared in the same manner as in the first method.

Then the prepared recombinant vector is introduced into unfertilized eggs, fertilized eggs or embryos, preferably unfertilized eggs of the mollusk. This may be carried out, for example, as follows: A vector solution having a concentration of about 100 to 200 mg/ml is placed in a petri dish or well and the unfertilized eggs, fertilized eggs or embryos are immersed therein. The eggs or embryos thus immersed are then keenly injured only in a moment with a micropipet for microinjection (not restricted thereto) so as to inject the vector solution into the eggs or the embryos. At this time, it is important to form a hole by injuring without bursting the cells or lethally injuring the cells. Individuals may be obtained from the eggs or embryos into which the recombinant vector was introduced by the same method as in the first method, and a transgenic line may be established by selecting the transgenic individuals as in the first method.

Individuals may be obtained from unfertilized eggs by carrying out artificial fertilization immediately after or simultaneously with the above-mentioned operation, and then developing the individuals therefrom as in the first method.

### EXAMPLES

The present invention will now be described by way of examples. It should be noted that the present invention are not restricted to the examples below.

### Reference Example 1 Preparation of Transgenic Pinctada fucata Martensii into which Human Interferon α Gene is Introduced

Human or mouse interferon α gene (commercially available from BBL and RDS, respectively) was inserted into an adenovirus vector (commercially available from TAKARA SHUZO, Takara Adenovirus Expression Vector Kit) to obtain recombinant vectors. This operation was carried out concretely as follows: Each of the above-mentioned commercially available interferon α genes was inserted into the Swa I site of a cosmid vector pAxCAwt (44,741 bp), Niwa, M. et al., (1991) Gene 108, 193, this cosmid vector is included in the above-mentioned commercially available Adenovirus Expression Vector Kit). The cosmid vector having the inserted gene and the above-mentioned commercially available adenovirus-derived DNA-TPC (Miyake, S. et al., (1996), Proc. Natl. Acad. Sci. USA 93 1320) digested with the above-mentioned restriction enzyme were co-transfected into 293 cells (human fetal kidney cell, commercially available from DAINIPPON PHARMACEUTICAL CO., LTD). The 293 cells were cultured in 10% FCS-containing DMEM medium under 5% CO₂ at 37° C. until 100% confluency is achieved, and 10 μg of the above-mentioned cosmid vector DNA and 5 μg of the restriction enzyme-treated DNA-TPC were mixed on a petri dish with a diameter of 6 cm. The transfection was carried out by the calcium phosphate method. The cells after the co-transfection were cultured at 37° C. under 5% CO₂ for 24 hours, and the fragment of grown recombinant adenovirus was recovered. The collected fragment was injected into ovaries of Pinctada fucata Martensii in an amount of 100 to 200 mg DNA/ovary. Sperms (twice amount of eggs) were mixed with the eggs in a test tube to carry out fertilization. The resulting eggs were cultured in sea water at 25° C. for 24 days to obtain young shells. In 31 young shells among 200 young shells, fluorescence (FITC) of a DNA probe for detecting interferon gene was observed. The DNAs of these young shells were purified and existence of the sequence was confirmed with the same DNA probe. These shells were continued to be cultivated.

It has been reported that adductor muscle is colored in red by virus infection. In 18 adult shells among 20 shells which were confirmed to have the interferon gene, this change of color to red was not observed.

On the other hand, each of the recombinant adenovirus vectors obtained as described above was transfected into Hela cells and grown therein. The transfection into Hela cells and proliferation of the virus were carried out by the method described in Nature 1995, 374(6523):660-662. The recombinant vector DNA was recovered from the Hela cells by a conventional method and the obtained DNA was dissolved in 10 mM Tris-HCl (pH7.5), 1 mM EDTA or 20 mM potassium phosphate, 3 mM potassium citrate and 2% PEG-6000 (pH7.5) to a concentration of 50 to 100 μg DNA/ml to obtain a solution for microinjection. This solution was microinjected into ovaries of female Pinctada fucata Martensii and testes of male Pinctada fucata Martensii. The amount of the injected solution was 100 μg in terms of DNA per site, and the solution was injected from three sites per ovary or testis. Twenty four to forty eight hours later, artificial fertilization was performed using sperms from the testes and mature eggs from the ovaries. The artificial fertilization was carried out concretely as follows: Testis was dissected from each male Pinctada fucata Martensii and ovary was dissected from each female Pinctada fucata Martensii, and sperms and eggs were taken out therefrom into a test tube at a ratio of 1:2, followed by mixing the sperms and the eggs. The obtained fertilized eggs were incubated in sea water for 2 to 3 weeks at 25° C. to obtain individuals of Pinctada fucata Martensii of the first generation from the fertilized eggs. Total DNAs were collected from the gonad of each of the obtained Pinctada fucata Martensii, and Southern blotting was carried out by a conventional method (Bio Experiments Illustrated, supra) using human interferon α gene as a probe. Further, total mRNAs were collected from block of visceral organs and from adductor muscle cells, and Northern blotting was carried out by a conventional method (Bio Experiments Illustrated, supra) using human interferon α gene as a probe.

Using mature eggs and sperms collected from male and female individuals which were Southern blot positive and Northern blot positive, artificial fertilization was carried out in the same manner as described above, and individuals of the second generation were obtained as described above. By carrying out Southern blot and Northern blot as described above, 3 lines of transgenic Pinctada fucata Martensii into which human interferon cc gene was introduced were selected.

The thus prepared transgenic Pinctada fucata Martensii and the conventional Pinctada fucata Martensii were cultivated for 180 days in a sea region which was thought to be contaminated with viruses and in a sea region which was thought to be not contaminated with viruses judging from the state of cultivated Pinctada fucata Martensii, and the lethalities of the groups were compared. The results are shown in Table 1.

TABLE 1
Effect of Transfection of Human Interferon α Gene
(cultivated for 360 days)

Lethality (%)

| Pinctada fucata | Non-contaminated | Contaminated Sea |
|---|---|---|
| Martensii | Sea Water | Water |
| Conventional Species | 10 | 95 |
| Line 1 | 6 | 30 |
| Line 2 | 1 | 35 |
| Line 3 | 7 | 32 |

As apparent from Table 1, while the lethalities of the transgenic Pinctada fucata Martensii according to the present invention (Lines 1-3) in the non-contaminated sea region were similar to that of the conventional species, the lethalities of the former in the contaminated sea region were much lower than that of the conventional species. These results clearly indicate the lethality-decreasing effect by transfection of human interferon α gene.

### Reference Example 2 Preparation of Transgenic Pinctada fucata Martensii into which Human Interferon β Gene is Introduced

The operations as in Reference Example 1 were repeated except that human interferon β gene (commercially available from BBL, HIGASHI, Y. et al. (1983) J. Biol. Chem. 258:92) was used in place of human interferon α gene, and that a 5′-end fragment of the human interferon β gene, which fragment has a size of 40 to 50 bp and labeled with fluorescent FITC was used as the probe in the Southern blot and Northern blot, to prepare transgenic Pinctada fucata Martensii (second generation) into which human interferon β gene was introduced.

The effectiveness of the transfection of the human interferon β gene was examined in the same as in Reference Example 1. The results are shown in Table 2.

TABLE 2
Effect of Transfection of Human Interferon β Gene
(cultivated for 180 days)

Lethality (%)

| Pinctada fucata | Non-contaminated | Contaminated Sea |
|---|---|---|
| Martensii | Sea Water | Water |
| Conventional Species | 11 | 94 |
| Line 1 | 10 | 28 |
| Line 2 | 14 | 22 |
| Line 3 | 12 | 15 |

As in Reference Example 1, the effect of the transfection of human interferon β gene was clearly observed.

### Example 1 Preparation of Pinctada fucata Martensii Having Green Fluorescent Protein (GFP) Gene (1)

Full length GFP gene (Science 1994, 263:802-805; GenBank No. U53602, commercially available from WAKO PURE CHEMICAL INDUSTRIES, LTD.) was inserted into the adenovirus vector in the same manner as in Reference Example 1 (the cells used for the growth of the virus were 293 cells). The obtained GFP gene-containing recombinant vector was dissolved in TE buffer to a concentration of 100 mg/ml, and the solution was microinjected into the ovaries of Pinctada fucata Martensii as in Reference Example 1. Thereafter, the same operations as in Reference Example 1 were repeated (except that the probe used in the Southern blot and Northern blot was GFP gene) to prepare transgenic Pinctada fucata Martensii(second generation).

Various tissues of the obtained transgenic Pinctada fucata Martensii were examined for fluorescence with a fluorescence microscope. The results are shown in Table 3 below. In Table 3, “+” means that fluorescence was observed, and the more the number of “+”, the stronger the emitted fluorescence.

TABLE 3
Tissues in Which Emitted Fluorescence Was Observed

| Transgenic | Block of | | | | |
|---|---|---|---|---|---|
| Pinctada fucata | Bisceral | | Adductor | | |
| Martensii | Organs | Atrium | Muscle | Mantle | Leg |
| Line 1 | +++ | ++ | ++ | +++ | ++ |
| Line 2 | + | + | + | + | +++ |
| Line 3 | +++ | +++ | ++ | +++ | ++ |

### Example 2 Preparation of Pinctada fucata Martensii Having Green Fluorescent Protein (GFP) Gene (2)

Prism protein of pearl shell is an important protein constituting pearls. In this Example, it was tried to make pearls emit fluorescence by themselves by fusing the prism protein gene with the green fluorescence protein gene.

Prism protein gene of Pinctada fucata Martensii (Nature 1997, 387(6633):563-564, hereby incorporated by reference); GenBank No. D860/3) together with its upstream region from its initiation codon to an upstream site thereof by 5 kb was cloned, and GFP gene was fused with the promoter, followed by inserting the resultant to an adenovirus vector. The fusion gene comprising the prism protein gene (containing the promoter) and the GFP gene was prepared by the method described by M. Chalfie et al., Science 1994, 263:802-805 hereby incorporated by reference). That is, at 10 nt from the initiation codon of the above-mentioned prism protein gene including the upstream region from the initiation codon by 5 kb, a linker (polyT) with a size of 9 nt, 10 nt or 11 nt was ligated. On the other hand, to the 5′-end of a commercially available GFP gene, polyA linker with a size of 9 nt, 10 nt or 11 nt was ligated. These DNA fragments were hybridized to obtain a fusion gene. Using restriction enzymes NHe I and Eco RI, the obtained fusion gene was inserted into the same adenovirus vector as used in Reference Example 1. Thereafter, in the same manner as in Reference Example 1, transgenic Pinctada fucata Martensii were obtained into which the fusion gene comprising the prism protein gene and the GFP gene was introduced. The mantle tissues of the obtained transgenic Pinctada fucata Martensii were observed with a fluorescence microscope to confirm emission of fluorescence.

The transgenic Pinctada fucata Martensii obtained in this Example contained the GFP gene at a site downstream of the promoter and the structural gene of the prism protein gene, and emission of fluorescence by expression of the fusion gene was observed. Therefore, if pearls are produced by these transgenic Pinctada fucata Martensii, it is thought that pearls which emit fluorescence are formed because GFP is fused with the prism protein constituting the pearls.

### Example 3 Preparation of Pinctada fucata Martensii Having Green Fluorescent Protein (GFP) Gene (3)

Mantle protein is an important protein constituting pearls like prism protein. The same operations as in Example 2 were repeated except that the mantle protein gene (Nature 1997, 387(6633):563-564 (hereby incorporated by reference), GenBank No. 86074) to prepare transgenic Pinctada fucata Martensii into which a fusion gene comprising mantle protein gene and the GFP gene was introduced. The fusion of the Pinctada fucata Martensii mantle protein gene and the GFP gene, and the insertion of the fusion gene into the adenovirus vector were carried out concretely as follows: As in Example 2, polyT with a size of 9 nt, 10 nt or 11 nt was ligated to the 15 nt from the initiation codon of a DNA fragment including the upstream region of the initiation codon of the mantle protein to an upstream site thereof by about 5 kb. On the other hand, polyA with a size of 9 nt, 10 nt or 11 nt was ligated to the GFP gene as used in Example 2. These DNA fragments were hybridized to prepare a fusion gene. Using the thus obtained fusion gene, the same operations as in Example 2 were repeated to obtain transgenic Pinctada fucata Martensii.

The mantle tissues of the obtained transgenic Pinctada fucata Martensii were observed with a fluorescence microscope to confirm emission of fluorescence.

The transgenic Pinctada fucata Martensii obtained in this Example contained the GFP gene at a site downstream of the promoter and the structural gene of the mantle protein gene, and emission of fluorescence by expression of the fusion gene was observed. Therefore, if pearls are produced by these transgenic Pinctada fucata Martensii, it is thought that pearls which emit fluorescence are formed because GFP is fused with the mantle protein constituting the pearls.

### Example 4 Introduction of GFP Gene or LacZ Gene by Promoter Trap Method

Basically, the method by I. A. Hope, Development, Vol. 113, 339-408 (1991) (hereby incorporated by reference) was followed. Firstly, by the method described in Example 1, GFP gene (10 to 50 mg DNA/gonad) was introduced into testes and ovaries, and then fertilization was carried out in test tubes. The eggs were cultured in sea water at 25° C. for 3 weeks to obtain young shells. About 5 to 15% of the young shells showed the coloring characteristic to GFP. These were cultivated in a cultivation bath for 12 months to obtain adult shells. The adult shells were anatomized and the organs which especially well colored were separated, followed by extraction of DNAs from the organs. In the extracted DNAs, the promoter controlling expression of the GFP gene was analyzed. Promoters of protein synthetases and of enzymes in the adductor muscle were trapped.

At a site downstream of the newly isolated sequences containing the promoter region, GFP gene or LacZ gene was inserted, and each of the obtained genes was inserted into the Swa I restriction site (between the cytomegalovirus enhancer sequence and rabbit β-globin polyA signal) of an expression vector pAxCAwt (TAKARA Adenovirus Expression Vector Kit). A solution of each of the obtained recombinant vectors with a concentration of about 100 to 200 mg/ml was prepared in a petri dish, and non-fertilized eggs were immersed therein. The eggs thus immersed are then keenly injured only in a moment with a micropipet for microinjection so as to inject the vector solution into the eggs. Thereafter, the same operations as in Reference Example 1 were repeated to obtain transgenic Pinctada fucata Martensii. The amount of expression of each gene was spectroscopically determined.

The obtained transgenic Pinctada fucata Martensii were examined for self emission of fluorescence (in case of introducing GFP gene) or for staining by coloring substrate XG (in case of introducing LacZ gene), and emission of fluorescence or staining was confirmed in various tissues of Pinctada fucata Martensii.

### INDUSTRIAL AVAILABILITY

By the present invention, transgenic mollusks having the desired properties may be provided. Thus, for example, industrially useful mollusks such as Pinctada fucata Martensii which yield colored pearls may be provided.

1. A transgenic pearl producing mollusk comprising a recombinant vector comprising a nucleic acid construct that contains a promoter that is operably linked to a foreign gene encoding a pigment protein or encoding an enzyme that catalyzes a reaction that forms a pigment, wherein said foreign gene is fused with a prism protein gene or a mantle protein gene, and wherein expression of the nucleic acid construct in the mollusk results in emission of fluorescence in mantle tissue of the mollusk.

2. A method for producing the transgenic pearl producing mollusk according to claim 1, comprising
introducing into unfertilized eggs, fertilized eggs or embryos of a mollusk to be transformed a recombinant vector comprising a nucleic acid including a promoter having promoter activity in said mollusk operably linked to said foreign gene encoding a fluorescent pigment protein or encoding an enzyme that catalyzes a reaction that forms a fluorescent pigment, wherein said foreign gene is located at a downstream region of said promoter and functionally linked to said promoter, and wherein said foreign gene is fused with a prism protein gene or a mantle protein gene;
developing said unfertilized eggs, fertilized eggs or embryos to individuals; and
selecting therefrom (an) individual(s) which express(es) said foreign gene.

3. A method for producing the transgenic pearl producing mollusk according to claim 1, comprising
microinjecting a recombinant vector which comprises a promoter operably linked to said foreign gene encoding a fluorescent pigment protein or encoding an enzyme that catalyzes a reaction that forms a fluorescent pigment, wherein said foreign gene is fused with a prism protein gene or a mantle protein gene, into a gonad of a male and/or a female mollusk;
crossing said male and female to produce individuals of first generation; and
selecting therefrom an individual which expresses said foreign gene.

4. The transgenic pearl producing mollusk of claim 1, wherein the foreign gene encodes Green Fluorescent Protein (GFP).

5. The transgenic pearl producing mollusk of claim 1, wherein the promoter has promoter activity in mollusk cells and is located at an upstream region of the foreign gene.

6. The transgenic pearl producing mollusk of claim 1, wherein the vector is an animal cell vector.

7. The transgenic pearl producing mollusk of claim 6, wherein the animal cell vector is an adenovirus vector or a retrovirus vector.

8. The transgenic pearl producing mollusk of claim 1, wherein the promoter is selected from the group consisting of adenoviral gene promoter and prism protein gene promoter.
